(19) United States
(12) Patent Application Publication
(10) Pub. No.: US 2023/0154601 A1
(43) Pub. Date: May 18, 2023

(54) COMPUTER-IMPLEMENTED METHOD FOR MANAGING A DIGITAL INFORMATION EXCHANGE RELATED TO RESCUE OPERATIONS, AND METHOD FOR COORDINATING EMERGENCY PHYSICIANS AVAILABLE IN AN EMERGENCY PHYSICIAN POOL

(71) Applicant: peiker Holding GmbH, Bad Homburg (DE)

(72) Inventors: Sebastian TIMME, Berlin (DE); Erik FERNHOLZ, Berlin (DE)

(21) Appl. No.: 18/055,955

(22) Filed: Nov. 16, 2022

(30) Foreign Application Priority Data
Nov. 16, 2021 (DE) 102021129885.7

(51) Int. Cl. G16H 40/20; G16H 40/67; G08B 25/00

(52) U.S. Cl. CPC G16H 40/20; G16H 40/67; G08B 25/006

(57) ABSTRACT

The present disclosure relates to a computer-implemented method for managing a digital information exchange in connection with rescue operations, the method being implemented in an electronic organization system (2), Furthermore, the present disclosure relates to a method for coordinating emergency physicians (501; 601) available in an emergency physician pool.

### CROSS-REFERENCES TO RELATED APPLICATION

This application claims the benefit of German Application No. 102021129885.7, filed Nov. 16, 2021.

### BRIEF DESCRIPTION OF THE DRAWINGS

FIG. 1 shows a schematic representation of a deployment scenario, in which the computer-implemented method according to one example for managing a digital information exchange in connection with rescue operations is described and in which the method according to one example for coordinating available emergency physicians in an emergency physician pool is described.

### DETAILED DESCRIPTION

The present disclosure relates to a computer-implemented method for managing a digital information exchange in connection with rescue operations according to the preamble of claim 1. Furthermore, the present disclosure relates to a computer-implemented method for the coordination of emergency physicians available in a pool of emergency physicians according to claim 11.

A method for operating a remote emergency physician system is known from DE 10 2020 109 040 A1, which comprises the following steps:
filming a patient with a portable digital camera carried by a user;
generating a collection of essential features of a patient on the basis of the digital images acquired by the filming, the collection of essential features being generated in particular from a face of the patient and the collection being generated by the portable digital camera or by at least one of the routers or by the server;
comparing the collection of the essential features of the patient with the digital images continuously recorded in the vehicle by an on-board digital camera by means of an algorithm, the algorithm being implemented in the digital cameras of the vehicle or in the router or in the server;
starting a transfer of the digital images recorded in the vehicle to a display screen of a monitoring module as soon as the result of the comparison shows that the patient already detected by the portable digital camera is detected by the digital camera installed in the vehicle.

It is one object of the present disclosure to propose a computer-implemented method for managing a digital information exchange in connection with rescue operations, and also a method for coordinating emergency physicians available in an emergency physician pool, by means of which the available emergency physician or available emergency physicians can be deployed more efficiently.

This object is achieved by the features of claim 1 and claim 11 respectively. The respective dependent claims contain advantageous and expedient extensions.

In the computer-implemented method according to one example of the present disclosure for managing a digital information exchange in connection with rescue operations, which is implemented in an electronic organization system,
which is connected to at least one control center via a communication link,
which is connected via a communication link to at least one ambulance and to at least one emergency paramedic assigned to the respective ambulance and
which is connected via a communication link to at least one emergency physician, in particular independently of the latter's location and speed of travel,
wherein the electronic organization system is notified for each connected emergency physician of a deployment status and an emergency physician assignment to an indications catalog,
wherein communication data, in particular as audio data and/or video data and/or location data and/or status data and/or control data, are exchanged via the electronic organization system depending on the situation.

This makes it possible for one emergency physician to handle multiple rescue operations, at least temporarily. This also makes it possible for an emergency physician with specialist knowledge to be recruited for handling the operation. Furthermore, it also enables an emergency physician who is driving towards a rescue operation or away from a rescue operation to be recruited to handle a rescue operation, at least temporarily.

The computer-implemented method also provides for
an emergency medical consultation to be initiated by the emergency paramedic via the electronic organization system, wherein the electronic organization system, on the basis of geocoordinates of the ambulance located at a particular accident location and, in particular, on the basis of an indication, automatically establishes a data connection to the emergency physician, who is selected as suitable for an emergency medical consultation with regard to the criteria of deployment status and emergency physician assignment and/or with regard to the criterion of shortest distance to the accident site,
at the same time as initiating the emergency medical consultation, the electronic organization system to be informed by the control center about the initiation of the emergency medical consultation and the control center to be granted partial or complete access to the communication between the emergency medical paramedic and the selected emergency physician and/or specialist physician by the organization system. This automatically enables a targeted and rapid selection of a suitable emergency physician, wherein despite the high degree of automation the control center remains informed without any time delay and can therefore intervene in special cases.

The computer-implemented method also provides for
an alarm to be issued to by the control center in the event of an accident reported to the control center via the organization system,
an alarm confirmation to be sent by the emergency paramedic to the control center via the organization system, and confirmation of the arrival at the accident site to be sent by the emergency paramedic via the organization system. This means that the computer-implemented method can also be used to send a conventional alarm to the emergency physician. This makes it easier to change over to the computer-implemented method, as this allows for a gradual modernization.

It is also provided that a further deployment criterion for each emergency physician can be created by the fact that the organization system automatically determines whether the respective emergency physician is connected to the electronic organization system from their workstation via a fixed router or from a position en route by means of a mobile router. Thus, the computer-implemented method can take into account other factors such as maximum possible data rate or temporary connection failure.

It can happen that the data rate is reduced due to faults in the mains supply. In the event of a reduced data rate, i.e. one that is insufficient for the transmission of all data available for transmission, the organization system specifies a prioritization for the data transmission, wherein it is provided in particular that voice communication is given the highest priority and that the transmission of the patient's vital signs data is given the second highest priority, with the transmission of a live video preferably given the lowest priority. In this case, the computer-implemented method can also provide for the prioritization to be modified by the emergency physician. This allows the prioritization to be optimally adapted to the individual deployment scenario.

In addition, the organization system can receive commands from the emergency physician or from the specialist physician, in particular by means of voice input or menu selection, for the execution of programmable routines by means of which rescue workers and/or auxiliary specialists and/or other specialists are automatically integrated into the organization system and are supplied with the available data, wherein the specialists that are integrated are in particular specialist physicians. This means that, in particular, a time advantage can be gained in the rescue process. Furthermore, such a degree of automation enables the emergency physician and/or the specialist physician to be only minimally distracted from their medical tasks by administrative activities.

The electronic organization system can also be equipped with a central computing unit and the electronic organization system can be equipped with terminal devices, which are each connected to it via at least one bidirectional communication path. This allows the entire process to be controlled centrally.

Furthermore, the computer-implemented method provides for
coordinates of a current location of the emergency physician to be processed as control data for the deployment criterion “travel time to the accident site” and/or
a number of ongoing consultations involving the emergency physician to be processed as control data for the deployment criterion “workload of the emergency physician” and/or
a designation of the medical specialty of the emergency physician to be processed as control data for the deployment criterion “specialization”. By comparing such control data, the most suitable emergency physician can be determined quickly and easily from a large number of available emergency physicians. As a result, restrictive constraints from a predetermined alarm catalog for the remote emergency physician can also be used to select the appropriate emergency physician.

It is also provided for a static workplace of the emergency physician and/or a mobile workplace of the emergency physician to be designed such that it is multisession-capable, so that parallel connections to multiple accident sites are established for the emergency physician if required. In this way, emergency care can be ensured at each of the accident sites, even in the event of an undersupply of emergency physicians.

Furthermore, a prioritization of the transmission of the various data types, such as in particular the data types of voice data and vital signs data and image data and live video data, can be carried out in such a way that, depending on a possible transmission quality and/or transmission rate or depending on, for example, a transmission quality and/or transmission rate to be expected along a route, e.g. of a journey from the accident site to the hospital, the prioritization of the data types is automated or can be both activated and deactivated by the emergency paramedic or by the emergency physician or the control center, and the data types are prioritized for transmission in particular in a prioritization order of voice data, vital signs data of the accident victim, image data, and live video data. This means that the emergency physician can ensure basic care provision despite difficult conditions.

It may also be possible for the prioritization order to be ultimately determined by the emergency physician. Depending on the medical requirements, the physician can switch between, for example, transmission of voice data and vital signs data of the accident victim or, for example, also briefly prioritize the exclusive transmission of live video data if this is required for diagnosis, for example.

In the method according to one example of the present disclosure for coordinating emergency physicians available in an emergency physician pool,
the emergency physician pool comprises at least one “mobile emergency physician”, whose emergency ambulance is equipped with means of communication such that the emergency physician can communicate with the emergency paramedic already at an accident site when approaching the site,
the emergency physician pool comprises at least one “remote emergency physician”, whose base is equipped in such a way that the emergency physician can communicate with the emergency paramedic already at the accident site,
wherein each emergency physician
   is assigned a first characteristic value, which indicates their emergency physician status, namely “mobile emergency physician” or “remote emergency physician”,
   is assigned a second characteristic value, which indicates their deployment status, namely “available” or “not available”,
   is assigned a third characteristic value, which indicates the indications from an indications catalog for which the emergency physician is a specialist,
wherein an electronic organization system is used to make a selection on the basis of the characteristic values, on the basis of an accident report and on the basis of information provided by the emergency paramedic,
   which is either sent to the control center as a proposal for an emergency physician to be commissioned from the emergency physician pool
   or by means of which an emergency physician from the emergency physician pool is automatically assigned.

This also makes it possible for an emergency physician with specialist knowledge to be consulted for the treatment. Furthermore, it also enables an emergency physician who is driving towards a rescue operation or away from a rescue operation to be recruited to handle a rescue operation, at least temporarily.

It is also provided, once a “mobile emergency physician” is selected for assignment, that the location of all “mobile emergency physicians” will be recorded as a fourth parameter and that the selection of the emergency physician will also depend on a calculated journey time to an accident site.

It is also provided that a means of transport available to the “mobile emergency physicians”, which can be designed in particular as a road vehicle or watercraft or aircraft, is recorded as a fifth characteristic value. This will further improve the automatic selection of a suitable emergency physician.

It is also provided for a static workplace of the emergency physician and/or a mobile workplace of the emergency physician to be designed such that it is multisession-capable, so that parallel connections to multiple accident sites are established for the emergency physician if required. In this way, emergency care can be ensured at each of the accident sites, even in the event of an undersupply of emergency physicians. Accordingly, it is also provided that in the case of parallel handling of different emergencies by the same emergency physician, a screen display of each emergency is adapted automatically or by the emergency physicians themselves, so that different amounts of data are displayed for each emergency according to the emergency situation. This makes it possible to reduce the amount of information without loss of quality, wherein, for example, in an emergency involving straightforward patient transport only the vital signs data of the patient is displayed.

Finally, it is also possible to determine the second characteristic value from an electronic duty roster or to verify it by means of an electronic duty roster. According to the first variant, the second characteristic value can easily be extracted from an existing planning system. According to the second variant, that is, if the deployment status is determined, e.g. by using automatic status detection or using a status set by the emergency physician or remote emergency physician themselves, the plausibility of the existing status can be easily checked so that incorrect information can be detected automatically.

For the purposes of the present disclosure, management of a digital information exchange means controlling and monitoring this digital information exchange.

Additional details of the present disclosure are described in the drawing on the basis of exemplary embodiments shown schematically.

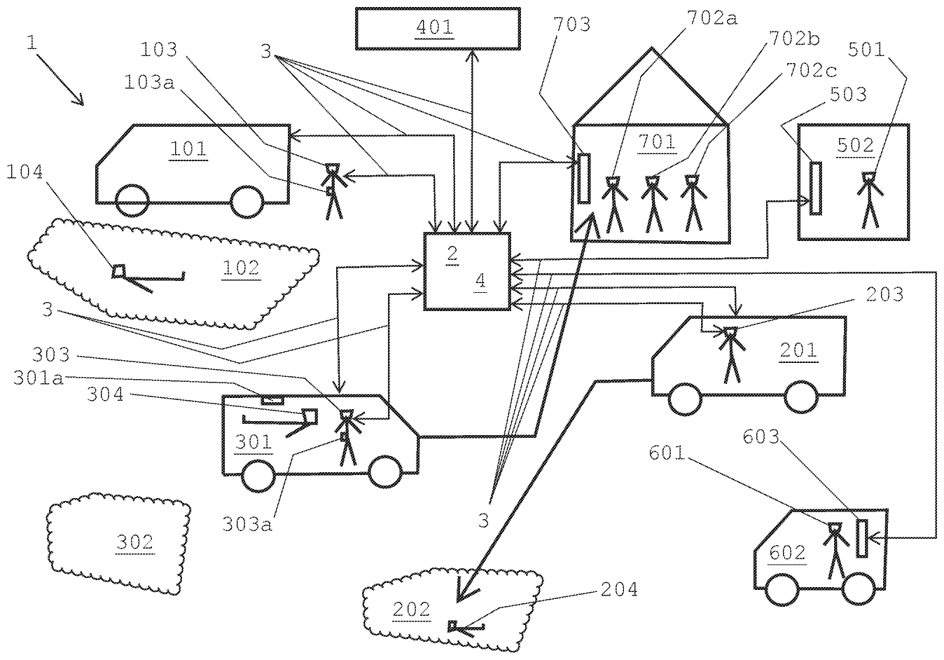

FIG. 1 shows a schematic deployment scenario 1, in which the computer-implemented method according to one example of the present disclosure for managing a digital information exchange in connection with rescue operations is described, and in which the method according to one example of the present disclosure for coordinating available emergency physicians in an emergency physician pool is described.

The deployment scenario 1 shows a first ambulance 101 from a plurality of ambulances, which is located at a first accident site 102. The ambulance 101 is assigned a first emergency paramedic 103, who is taking care of a first accident victim 104 outside the ambulance 101.

The deployment scenario 1 shows a second ambulance 201 from the plurality of ambulances, which is on the way to a second accident site 202. The second ambulance 201 is assigned a second emergency paramedic 203. At the accident site 202 there is a second accident victim 204.

The deployment scenario 1 shows a third ambulance 301 from the plurality of ambulances, which is on the way back from a third accident site 302. The ambulance 301 is assigned a third emergency paramedic 303, who is taking care of a third accident victim 304 in the ambulance 301.

The deployment scenario 1 also shows a control center 401.

In addition, the deployment scenario 1 shows a first emergency physician 501 who is available for deployment at a fixed first location 502, and a second emergency physician 601 who is travelling in an emergency ambulance 602 and is available for deployment. The first emergency physician 501 is also referred to as a remote emergency physician or static emergency physician, and the second emergency physician 601 is also referred to as a mobile emergency physician.

The deployment scenario 1 also shows a hospital 701, in which a plurality of specialist physicians 702a, 702b and 702c are on duty.

Finally, the deployment scenario 1 schematically shows an electronic organization system 2 in which the method for managing a digital information exchange in connection with rescue operations is implemented.

The electronic organization system 2 is connected via communication links 3 to the control center 401, to the first ambulance 101 and the first emergency paramedic 103, to the second ambulance 201 and the second emergency paramedic 203, to the third ambulance 301 and the third emergency paramedic 303, to the first emergency physician 501 via a terminal 503, to the second emergency physician 601 via a terminal 603, and to the specialist physicians 702a-702c via a terminal 703.

The electronic organization system 2 is notified of a deployment status and an emergency physician assignment to an indications catalog for each connected emergency physician 501, 601.

In addition, communication data, in particular as audio data and video data and location data and status data and control data, is exchanged via the electronic organization system 2 depending on the situation.

In this case, in the illustrated deployment scenario 1, audio data, video data and status data are exchanged between the first emergency paramedic 103 and the first emergency physician 501 via the electronic organization system 2. As a result, the first emergency physician 501 can observe the first accident victim 104 by means of a bodycam 103a worn by the first emergency paramedic 103, is simultaneously in voice communication with the first emergency paramedic 103, and receives vital signs data of the first accident victim 102 transmitted as status data. For this purpose, for example, an ECG (not shown) is used, which is also connected wirelessly to the electronic organization system.

In this case, the second ambulance 201 has received geocoordinates of the second accident site 202 via the electronic organization system 2 on the basis of an emergency call received in the control center 401 and is on its way to the second accident victim 204. Furthermore, on the basis of the data “life-threatening injury” and “infant” reported to the control center 401 regarding the 5 second accident victim 204, the electronic organization system 2 has alerted the second mobile emergency physician 601 as he/she can reach the second accident site 202 the fastest and as the suspected severity of the accident requires an emergency physician on site. In this case, the time required to arrive at the second accident site 202 was determined by the electronic organization system 2 on the basis of the geocoordinates of the second accident site 202 and on the basis of the geocoordinates that the emergency ambulance 602 of the second emergency physician 601 had at the time of the emergency call. In addition, on the basis of the reported data regarding the accident victim, the electronic organization system 2 has already alerted one of the specialist physicians 702a-702c in the hospital 701, who can support the second emergency physician 602 via the terminal 703 available in the hospital 701. The emergency physician 601 can then connect to the emergency paramedic 203, who in the present scenario will arrive at the accident site 202 before the emergency physician 601, and guide the latter in the first-aid treatment. For this purpose, the emergency physician 601 uses the terminal 603.

The treatment of the third accident victim 304, who is in the third ambulance 301 on the way to the hospital 701, is supported via multisession by the first emergency physician 501, who by means of a camera 301a present in the third ambulance 301 or a bodycam 303a worn by the third emergency paramedic 303 can observe the third accident victim 304, is in voice communication with the third emergency paramedic 303, and receives vital signs data of the third accident victim 304 transmitted as status data. For this purpose, the multisession-capable terminal 503 is available at the first location 502.

The electronic organization system 2 offers the control center 401 the facility to track the communication data of the individual rescue operations, so that, for example, in the event of overloading of the rescue system the control center 401 can track and classify the ongoing rescue operations and intervene in a coordinated manner.

Both the emergency paramedics 103, 203, 303 and also the emergency physicians 501, 601, the emergency ambulances 101, 201, 301 and the emergency ambulance 602 are equipped with terminal devices which enable the described communication within the electronic organization system.

The electronic organization system 2 comprises a central computing unit 4. This is connected via the communication links 3, which are implemented as wireless, bidirectional communication channels, to the various terminal devices such as the bodycams 103a, 303a, the camera 301a, and the terminals 503, 603, 703.

An emergency physician pool includes the emergency physicians 501 and 601 as well as other emergency physicians not explicitly mentioned, which at district borders can also be mobile emergency doctors from other districts and which can also be hospital-based emergency doctors from any other districts.

It can also be provided that in the computer-implemented method for managing a digital information exchange in connection with ambulances a prioritization of the transmission of the various data types, such as in particular the data types of voice data and vital signs data and image data and live video data, is carried out in such a way that, depending on a possible transmission quality and/or transmission rate or depending on, for example, a transmission quality and/or transmission rate to be expected along a route, e.g. of a journey from the accident site to the hospital, the prioritization of the data types is automated or can be both activated and deactivated by the emergency paramedic or by the emergency physician or the control center, and the data types are prioritized for transmission in particular in a prioritization order of voice data, vital signs data of the accident victim, image data and live video data.

In the computer-implemented method it is provided that the prioritization order is ultimately determined by the emergency physician. This means that, if required due to low transmission quality and/or transmission rate, the physician can switch between the data types to provide the emergency paramedic with optimum support.

This also makes it possible for the emergency physician to virtually accompany one or more patient transports. If the emergency physician's terminal is operated in multisession mode, they can also virtually accompany multiple patient transports and make the prioritization of the data rates dependent, for example, on the condition of the individual patients, wherein a display of the vital signs data of the individual patients will usually be sufficient.

Although the present invention has been described with reference to preferred embodiments, workers skilled in the art will recognize that changes may be made in form and detail without departing from the spirit and scope of the invention.

### LIST OF REFERENCE SIGNS

1 deployment scenario
2 electronic organization system
3 communication link to 2
4 computing unit
101 first ambulance
102 first accident site
103 first emergency paramedic
103a bodycam belonging to 103
104 first accident victim
201 second ambulance
202 second accident site
203 second emergency paramedic
204 second accident victim
301 third ambulance
301a camera in 301
302 third accident site
303 third emergency paramedic
303a bodycam belonging to 303
304 third accident victim
401 control center
501 first emergency physician or remote emergency physician or static emergency physician
502 first location
503 multisession-capable terminal
601 second emergency physician or mobile emergency physician
602 ambulance belonging to 601
701 hospital
702a-702c specialist physician
703 terminal

1. A computer-implemented method for managing a digital exchange of information in connection with rescue operations, the method being implemented in an electronic organization system,
which is connected to at least one control center via a communication link,
which is connected via a communication link to at least one ambulance and to at least one emergency paramedic assigned to the respective ambulance, and
which is connected to at least one emergency physician via a communication link,
wherein the electronic organization system is notified of a deployment status and an emergency physician assignment to an indications catalog for each connected emergency physician,
wherein communication data, in particular as audio data and/or video data and/or location data and/or status data and/or control data are exchanged via the electronic organization system depending on the situation.

2. The computer-implemented method as claimed in claim 1, wherein
an emergency medical consultation is initiated by the emergency paramedic via the electronic organization system, wherein the electronic organization system, on the basis of geocoordinates of the ambulance located at a particular accident location and, in particular, on the basis of an indication, automatically establishes a data connection to the emergency physician, selected as suitable for an emergency medical consultation with regard to the criteria of deployment status and emergency physician assignment and/or with regard to the criterion of shortest distance to the accident site,
that, at the same time as initiating the emergency medical consultation, the control center is informed by the electronic organization system of the initiation of the emergency medical consultation, and that the control center is granted partial or complete access to the communication between the emergency medical paramedic and the selected emergency physician and/or specialist physician by the organization system.

3. The computer-implemented method as claimed in claim 1, wherein
an alarm is issued to the ambulance by the control center in the event of an accident reported to the control center via the organization system,
confirmation of the alarm issue by the emergency paramedic is sent to the control center via the organization system,
the arrival at the accident site is confirmed by the emergency paramedic via the organization system.

4. The computer-implemented method as claimed in claim 1, wherein a further deployment criterion for each emergency physician is created by the fact that the organization system automatically determines whether the respective emergency physician is connected to the electronic organization system from their workstation via a fixed router or from a position en route by means of a mobile router.

5. The computer-implemented method as claimed in claim 1, wherein the organization system can receive commands from the emergency physician or from the specialist physician, in particular by means of voice input or menu selection, for the execution of programmable routines by means of which rescue workers and/or auxiliary specialists and/or other specialists are automatically integrated into the organization system and are supplied with the available data, wherein the specialists that are integrated are specialist physicians.

6. The computer-implemented method as claimed in claim 1, wherein the electronic organization system comprises a central computing unit and that the electronic organization system comprises terminal devices connected to each of these via at least one bidirectional communication path.

7. The computer-implemented method as claimed in claim 1, wherein
coordinates of a current location of the emergency physician are processed as control data for the deployment criterion “travel time to the accident site” and/or
that a number of ongoing consultations involving the emergency physician is processed as control data for the deployment criterion “workload of the emergency physician” and/or
that a designation of the medical specialty of the emergency physician is processed as control data for the deployment criterion “specialization”.

8. The computer-implemented method as claimed in claim 1, wherein a static workplace of the emergency physician and/or a mobile workplace of the emergency physician is/are designed such that it is multisession-capable, so that parallel connections to multiple accident sites are established for the emergency physician if required.

9. The computer-implemented method as claimed in claim 1, wherein a prioritization of the transmission of the various data types is carried out in such a way that, depending on a possible transmission quality and/or transmission rate, the prioritization of the transmission of the various data types is automated or can be both activated and deactivated by the emergency paramedic or by the emergency physician or the control center, and the various data types are prioritized for transmission in particular in a prioritization order of voice data, vital signs data of the accident victim, image data and live video data.

10. The computer-implemented method as claimed in claim 9, wherein the prioritization order is ultimately implemented by the emergency physician.

11. A method for coordinating emergency physicians available in an emergency physician pool,
wherein the emergency physician pool comprises at least one mobile emergency physician, whose emergency ambulance is equipped with means of communication such that the emergency physician can communicate with the emergency paramedic already at an accident site when approaching the site,
wherein the emergency physician pool comprises at least one so-called “remote emergency physician”, whose base is equipped in such a way that the emergency physician can communicate with the emergency paramedic already at the scene of the accident,
wherein each emergency physician
is assigned a first characteristic value which indicates their emergency physician status, namely “mobile emergency physician” or “remote emergency physician”,
a second characteristic value is assigned which indicates their deployment status, namely “available” or “not available”,
a third characteristic value is assigned, which specifies the indications from an indications catalog for which the emergency physician is a specialist,
wherein an electronic organization system is used to make a selection on the basis of the characteristic values, on the basis of an accident report and on the basis of information provided by the emergency paramedic,
which is either sent to the control center as a proposal for an emergency physician to be assigned from the emergency physician pool
or by means of which an emergency physician from the emergency physician pool is automatically assigned.

12. The method as claimed in claim 11, wherein if a “mobile emergency physician” is selected for assignment, the location is recorded as a fourth characteristic value for all “mobile emergency physicians” and the selection of the emergency physician is influenced by a calculated travel time to an accident site.

13. The method as claimed in claim 12, wherein a means of transport available to the “mobile emergency physicians”, which is designed in particular as a road vehicle or watercraft or aircraft, is recorded as a fifth characteristic value.

14. The method as claimed in claim 11, wherein a static workplace of the emergency physician and/or a mobile workplace of the emergency physician is/are designed such that it is multisession-capable, so that parallel connections to multiple accident sites are established for the emergency physician if required.

15. The method as claimed in claim 11, wherein the second characteristic value is determined from an electronic duty roster or is verified by means of an electronic duty roster.

16. The computer-implemented method as claimed in claim 9, wherein the prioritization of the transmission of the various data types comprises a prioritization of the data types of voice data and vital signs data and image data and live video data.

17. The computer-implemented method as claimed in claim 1, wherein a prioritization of the transmission of the various data types is carried out in such a way that, depending on a transmission quality and/or transmission rate to be expected along a route, the prioritization of the transmission of the various data types is automated or can be both activated and deactivated by the emergency paramedic or by the emergency physician or the control center, and the data types are prioritized for transmission in particular in a prioritization order of voice data, vital signs data of the accident victim, image data and live video data.

18. The computer-implemented method as claimed in claim 17, wherein the route comprises a journey from the accident site to the hospital.

19. The computer-implemented method as claimed in claim 17, wherein the prioritization of the transmission of the various data types comprises a prioritization of the data types of voice data and vital signs data and image data and live video data.
